(19) United States
(12) Patent Application Publication
(10) Pub. No.: US 2026/0077145 A1
(43) Pub. Date: Mar. 19, 2026

(54) METHOD FOR LEAK TESTING IN CIRCUIT OF MEDICAL DEVICE, DEVICE, AND STORAGE MEDIUM

(71) Applicant: GE Precision Healthcare LLC, Waukesha, WI (US)

(72) Inventors: Shuiqing Yu, Wuxi (CN); Zheng You, Wuxi (CN)

(21) Appl. No.: 19/319,402

(22) Filed: Sep. 4, 2025

(30) Foreign Application Priority Data
Sep. 14, 2024 (CN) 202411290717.4

(51) Int. Cl. A61M 16/00

(52) U.S. Cl. CPC A61M 16/024; A61M 16/0087; A61M 2202/0007; A61M 2202/0241; A61M 2205/15; A61M 2205/3327; A61M 2205/3331; A61M 2205/50

(57) ABSTRACT

The present disclosure relates to the technical field of medical devices, and in particular to a method for leak testing in a circuit of a medical device, a device, and a storage medium, the circuit including a reservoir bag, the method including: pressurizing the circuit; determining a compliance characteristic of the reservoir bag; stopping pressurizing the circuit, and determining a wait time of the circuit on the basis of the compliance characteristic of the reservoir bag; and determining a change in gas pressure in the circuit before and after the wait time, thereby testing whether the circuit is leaking.

### CROSS-REFERENCE TO RELATED APPLICATIONS

This application is based on and claims priority to Chinese Application No. 202411290717.4, filed on Sep. 14, 2024, the entire contents of which is herein incorporated by reference.

### TECHNICAL FIELD

The present disclosure relates to the technical field of medical devices, and in particular to a method for leak testing in a circuit of a medical device, a device, and a storage medium.

### BACKGROUND

In the inspection of systems of medical devices, leak testing in circuits is an important type of leak detection. The problem of circuit leaks may result in reduced treatment effectiveness, and even endanger a patient's life. Therefore, it is critical to ensure that medical devices pass rigorous leak tests in a circuit before being put into use.

Currently, many medical devices use a pressure decay method for leak testing in a circuit, and in said method leakage is inferred by measuring a decrease in circuit pressure over time after the circuit is pressurized.

### SUMMARY OF THE INVENTION

The inventors have found that when the foregoing pressure decay method is used to perform leak tests in different circuits, the accuracy of the test results is often uncertain. The inventors have further found by research that the foregoing uncertainty often occurs in a circuit including a reservoir bag. The reason is that, as a gas-containing apparatus that can be inflated by pressurization, different reservoir bags have different deformation characteristics (or compliance characteristics) after being pressurized. Therefore, reservoir bags with different compliance characteristics have different influences on changes in pressure in a circuit during the pressure decay process, which may eventually cause a deviation in the results.

In view of this, the present disclosure provides a method for leak testing in a circuit of a medical device, a device, and a storage medium, which can adapt to reservoir bags of different types and sizes and improve the accuracy and reliability of the test.

According to an aspect of the present disclosure, there is provided a method for leak testing in a circuit of a medical device, the circuit comprising a reservoir bag, the method comprising: pressurizing the circuit; determining a compliance characteristic of the reservoir bag; stopping pressurizing the circuit, and determining a wait time of the circuit on the basis of the compliance characteristic of the reservoir bag; and determining a change in gas pressure in the circuit before and after the wait time, thereby testing whether the circuit is leaking.

In a possible implementation, determining the compliance characteristic of the reservoir bag comprises: testing a compliance of the reservoir bag; obtaining a compliance of a standard reservoir bag; comparing a compliance magnitude and/or a compliance similarity between the reservoir bag and the standard reservoir bag, and using a comparison result as the compliance characteristic of the reservoir bag.

In another possible implementation, determining the wait time of the circuit on the basis of the compliance characteristic of the reservoir bag comprises: obtaining a standard wait time corresponding to the standard reservoir bag; according to the compliance similarity between the reservoir bag and the standard reservoir bag, determining a corresponding adjustment factor by using a preset mapping model, the mapping model being used for indicating a correspondence between the compliance similarity and the adjustment factor; and adjusting the standard wait time according to the adjustment factor, and determining the adjusted standard wait time to be the wait time of the circuit.

In another possible implementation, determining the wait time of the circuit on the basis of the compliance characteristic of the reservoir bag comprises: obtaining a standard wait time corresponding to the standard reservoir bag, adjusting the standard wait time according to an adjustment factor, and determining the adjusted standard wait time to be the wait time of the circuit, and: when the compliance magnitude indicates that a hardness of the reservoir bag is higher than a hardness of the standard reservoir bag, shortening the standard wait time according to the adjustment factor; when the compliance magnitude indicates that a hardness of the reservoir bag is lower than a hardness of the standard reservoir bag, extending the standard wait time according to the adjustment factor; and when the compliance magnitude indicates that the hardness of the reservoir bag is equal to the hardness of the standard reservoir bag, keeping the standard wait time unchanged.

In another possible implementation, determining the wait time of the circuit on the basis of the compliance characteristic of the reservoir bag comprises: obtaining a standard wait time corresponding to the standard reservoir bag; when the compliance magnitude indicates that a hardness of the reservoir bag is higher than a hardness of the standard reservoir bag, according to the compliance similarity, determining a corresponding first adjustment factor by using a preset first mapping model, shortening the standard wait time according to the first adjustment factor, and determining the shortened standard wait time to be the wait time of the circuit; when the compliance magnitude indicates that a hardness of the reservoir bag is lower than a hardness of the standard reservoir bag, according to the compliance similarity, determining a corresponding second adjustment factor by using a preset second mapping model, extending the standard wait time according to the second adjustment factor, and determining the extended standard wait time to be the wait time of the circuit, wherein the second mapping model is different from the first mapping model; and when the compliance magnitude indicates that the hardness of the reservoir bag is equal to the hardness of the standard reservoir bag, keeping the standard wait time unchanged, and determining the standard wait time to be the wait time of the circuit.

In another possible implementation, testing the compliance of the reservoir bag comprises: monitoring an internal pressure of the reservoir bag in the process of pressurizing the circuit; and obtaining a curve of change in the internal pressure of the reservoir bag versus time to test the compliance of the reservoir bag.

In another possible implementation, comparing the compliance magnitude and/or the compliance similarity between the reservoir bag and the standard reservoir bag comprises: determining the compliance magnitude between the reservoir bag and the standard reservoir bag according to the compliance of the reservoir bag and the compliance of the standard reservoir bag; and/or, determining the compliance similarity between the reservoir bag and the standard reservoir bag according to a pressure change rate of the reservoir bag over time and a pressure change rate of the standard reservoir bag over time.

In another possible implementation, the pressure change rate of the reservoir bag over time comprises a plurality of measured values, the pressure change rate of the standard reservoir bag over time comprises a plurality of standard values, and determining the compliance similarity between the reservoir bag and the standard reservoir bag according to the pressure change rate of the reservoir bag over time and the pressure change rate of the standard reservoir bag over time comprises: determining standard values respectively corresponding to the plurality of measured values from among the plurality of standard values; and determining the compliance similarity between the reservoir bag and the standard reservoir bag by using a preset similarity calculation model according to the plurality of measured values and the standard values respectively corresponding to the plurality of measured values.

In another possible implementation, the compliance is negatively correlated with a hardness of the reservoir bag, and: when the compliance of the reservoir bag is less than the compliance of the standard reservoir bag, it is determined that the compliance magnitude indicates that a hardness of the reservoir bag is higher than a hardness of the standard reservoir bag; when the compliance of the reservoir bag is greater than the compliance of the standard reservoir bag, it is determined that the compliance magnitude indicates that a hardness of the reservoir bag is lower than a hardness of the standard reservoir bag; and when the compliance of the reservoir bag is equal to the compliance of the standard reservoir bag, it is determined that the compliance magnitude indicates the hardness of the reservoir bag is equal to the hardness of the standard reservoir bag.

In another possible implementation, the medical device comprises a ventilator device or an anesthesia machine device.

According to another aspect of the present disclosure, there is provided a medical device, comprising: a processor; a memory for storing processor-executable instructions, wherein the processor is configured to, when executing the instruction stored in the memory, implement the method provided in the first aspect or any one of the possible implementations of the first aspect.

According to another aspect of the present disclosure, there is provided a non-volatile computer-readable storage medium, having computer program instructions stored thereon, wherein the computer program instructions, when executed by a processor, implement the method provided in the first aspect or any one of the possible implementations of the first aspect.

According to another aspect of the present disclosure, there is provided a computer program product, comprising computer-readable codes or a non-volatile computer-readable storage medium loaded with computer-readable codes, wherein when the computer-readable codes are run in a processor of an electronic device, the processor in the electronic device performs the method provided in the first aspect or any one of the possible implementations of the first aspect.

The embodiments of the present disclosure provide a method for leak testing in a circuit of a medical device. In the method, a compliance characteristic of a reservoir bag included in a circuit is determined by pressurizing the circuit; a wait time of the circuit is determined on the basis of the compliance characteristic of the reservoir bag after pressurizing the circuit is stopped; and a change in gas pressure in the circuit before and after the wait time is determined, thereby testing whether the circuit is leaking. Therefore, the following technical effects are achieved: 1. Adaptability: The method can automatically adapt to reservoir bags of different types and sizes, and does not need additional configuration of system software, thereby simplifying the testing process. 2. Accuracy and high reliability: The wait time determined on the basis of the compliance characteristic of the reservoir bag, in combination with the change in the gas pressure before and after the wait time, ensures the accuracy and reliability of the results of leak testing in a circuit. 3. Robustness: The method can adapt to various reservoir bags, which ensures the consistency and robustness of test results under different conditions, thereby providing a strong guarantee for the safe operation of medical devices.

Other features and aspects of the present disclosure will become apparent from the following detailed description of exemplary embodiments provided with reference to the accompanying drawings.

### BRIEF DESCRIPTION OF THE DRAWINGS

The accompanying drawings, which are incorporated in and constitute a part of the specification, illustrate exemplary embodiments, features, and aspects of the present disclosure together with the description, and serve to explain the principles of the present disclosure.

FIG. 1 is a schematic structural diagram of an anesthesia ventilator device according to an embodiment of the present disclosure.

FIG. 2 shows a flowchart of a method for leak testing in a circuit of a medical device provided by an exemplary embodiment of the present disclosure.

FIG. 3 shows a curve graph of changes in a pressure value of a circuit versus time during testing provided by an exemplary embodiment of the present disclosure.

FIG. 4 shows a flowchart of a method for leak testing in a circuit of a medical device provided by another exemplary embodiment of the present disclosure.

FIG. 5 shows a flowchart of a method for leak testing in a circuit of a medical device provided by another exemplary embodiment of the present disclosure.

FIG. 6 shows a flowchart of a method for leak testing in a circuit of a medical device provided by another exemplary embodiment of the present disclosure.

### DETAILED DESCRIPTION

Various exemplary embodiments, features, and aspects of the present disclosure will be described in detail below with reference to the accompanying drawings. The same reference numerals in the drawings represent elements having the same or similar functions. While various aspects of the embodiments are illustrated in the accompanying drawing, the accompanying drawings are not necessarily drawn to scale unless specifically stated.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies. The terms “pixel” and “voxel” may be used interchangeably.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context. The word “exemplary” dedicated herein means “used as an example or an embodiment, or is illustrative”. Any of the embodiments illustrated herein as “exemplary” is not necessarily interpreted as being superior to or better than other embodiments.

In addition, in order to better illustrate the present disclosure, numerous specific details are given in the detailed description below. It should be understood by those skilled in the art that the present disclosure can be implemented without some of the specific details. In some examples, the methods, means, elements, and circuits well known to those skilled in the art are not described in detail in order to highlight the main idea of the present disclosure.

First, an execution subject of the embodiments of the present disclosure is introduced. A method for leak testing in a circuit of a medical device provided by the embodiments of the present disclosure may be applied to a medical device. The medical device may include a ventilator device or an anesthesia machine device, or may include an anesthesia ventilator device. The anesthesia ventilator device has both the function of the anesthesia machine device and the function of the ventilator device, and is a medical device that combines the characteristics of the two devices. The medical device will be described below by taking an anesthesia ventilator device as an example.

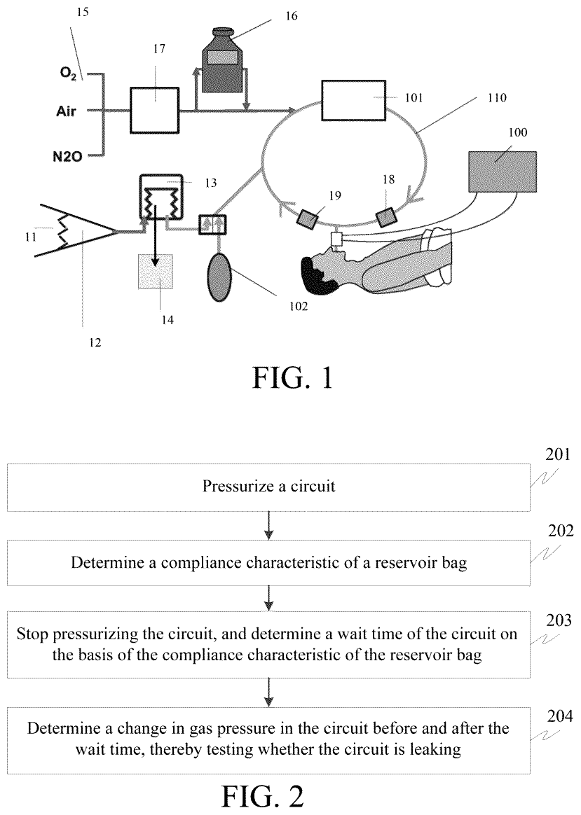

FIG. 1 is a schematic structural diagram of an anesthesia ventilator device 10 according to an embodiment of the present disclosure. As shown in FIG. 1, the anesthesia ventilator device includes: a driving gas source 11, a ventilation engine 12, a corrugated tube 13, an exhaust system 14, a breathing gas source 15, an anesthesia source 16, a mixer 17, an intake flow control valve 18, an exhalation flow control valve 19, a circuit 110, and conduits (not shown) connecting the components.

Optionally, the breathing gas source 15 is connected to the mixer 17; the breathing gas source 15 provides, for example, fresh gas, such as oxygen, air, laughing gas (nitrous oxide), etc., which are mixed in the mixer 17; the mixer 17 is further connected to the anesthesia source 16; after an anesthetic is added to the mixed gas, the mixed gas flows into the interior of the corrugated tube 13; and the interior of the corrugated tube is in communication with the respiratory tract of a subject.

Optionally, the ventilation engine 12 is connected to the driving gas source 11, the ventilation engine 12 may include a pressure reducing valve, a flow valve, a flow sensor, and a check valve (not shown) that are connected in sequence, and a driving gas (for example, air) of the driving gas source 11 flows into the check valve through the flow valve and the flow sensor. A pressure sensor may be integrated into the flow sensor, or may be present as a separate assembly in the ventilation engine 12.

Optionally, the check valve is connected to the corrugated tube 13, a pressurized gas flow (driving gas) flowing out from a gas hole of the valve body of the check valve pushes the outer wall of the corrugated tube 13 at the rear end, and after pressure is applied to the outer wall of the corrugated tube 13, it is equivalent to providing a driving force to drive the gas inside of the corrugated tube 13 into the respiratory tract of the subject. Since a magnetic damper is provided in the check valve, the gas hole can be gently opened and closed, thereby preventing the pressurized gas flow from impacting the corrugated tube 13 to cause vibration.

Optionally, the interior of the corrugated pipe 13 is further in communication with the exhaust system 14, and the exhaust system 14 is used for recovering anesthetic exhaust gas. For the implementations of the exhaust system 14 (such as an AGSS), reference may be made to the prior art, and they are not described in detail herein.

Optionally, the intake flow control valve 18 and the exhalation flow control valve 19 (for example, a diaphragm or a balloon valve) are disposed in a conduit near a intake port and a conduit near an exhalation port, respectively, and are used to control the gas flow and pressure of the intake and exhalation, wherein the intake flow control valve may be a solenoid valve.

During most of the inspiration stage of a patient's respiration, gas with the anesthetic is transmitted to the conduit through the intake flow control valve 18 and finally reaches the patient through the drive of the ventilation engine 12. In most of the expiration stage of the patient's respiration, the check valve 10 is closed, and exhaled gas passes through the conduit to the exhalation flow control valve 19 and is then discharged to the exhaust system 14.

The medical device may further include a monitoring device 100 for monitoring vital signs (respiratory rate) of the subject, gas pressure and the like, a carbon dioxide absorber 101, a manual control device such as a reservoir bag 102, etc. The fresh gas (driven by the driving gas) with the anesthetic enters the lungs of the subject through the circuit 110, and after the diaphragm muscle contracts, carbon dioxide is discharged in the circuit 110 and absorbed by the carbon dioxide absorber 101. The reservoir bag 102 plays a role similar to that of the ventilation engine 12. The medical device does not necessarily include all the components shown in FIG. 1. In addition, the medical device may further include components not shown in FIG. 1, which are set according to actual requirements and are not illustrated one by one herein.

The circuit 110 generally refers to a gas circuit system that is directly connected to the patient, including assemblies such as the reservoir bag 102. The reservoir bag is a part of the circuit of the medical device, and is used to store and distribute gas, thereby assisting the respiration process of the patient, and ensuring the flow and circulation of gas. In the embodiments of the present disclosure, the method for leak testing in a circuit of a medical device includes: pressurizing the circuit 110; determining a compliance characteristic of the reservoir bag; stopping pressurizing the circuit 110, and determining a wait time of the circuit 110 on the basis of the compliance characteristic of the reservoir bag; and determining a change in gas pressure in the circuit 110 before and after the wait time, thereby testing whether the circuit 110 leaks. In other words, in the above embodiments of the present application, on the basis of the inventors' finding that the classical leak testing in a circuit may be inaccurate due to the different compliance characteristics of the reservoir bags, the influences of the compliance characteristics of the reservoir bags on the test are comprehensively considered, and then the test is adjusted to improve the accuracy.

The method for leak testing in a circuit of a medical device provided by the embodiments of the present disclosure will be introduced below with reference to several exemplary embodiments.

Referring to FIG. 2, FIG. 2 shows a flowchart of a method for leak testing in a circuit of a medical device provided by an exemplary embodiment of the present disclosure. This embodiment is illustrated by taking the method being applied to the medical device as an example. The method includes the following steps:

Step 201: Pressurizing a circuit.

When the circuit of the medical device is pressurized, ensuring that the pressure reaches a required level is a critical step before leak testing. The process of pressurizing may be performed in a manual or automated manner to adapt to different operating environments and safety standards.

Manual pressurization allows an operator to achieve immediate control and precise adjustment of the pressure by physical means, such as by manually operating a pressurization device. The manual pressurization device may be any device in the prior art. This manner provides direct pressure feedback, enabling the operator to gradually increase the pressure in the circuit as needed.

For an application scenario in which high-precision control is pursued, a target pressure value may be set by means of an operation interface of the medical device. The medical device will automatically make adjustments to ensure that the pressure in the circuit reaches a preset target pressure value, such as 20 centimeter water column (cmH₂O).

In addition, pressurization may be performed in an automated manner. For example, a driving gas source may be connected to the circuit through a pipe and a valve. This typically involves opening a driving gas inlet valve to allow a desired gas (e.g., an external gas source) to flow into the circuit. Subsequently, the speed or flow rate of the gas flowing into the circuit is adjusted by adjusting a valve such as a flow control valve. Throughout the process of pressurizing, the change in the pressure in the circuit may also be monitored and fed back in real time by a pressure sensor to ensure that the pressure increases as expected and remains within a safe range. When the medical device is the device described in FIG. 1, the driving gas inlet valve may be a flow valve in the ventilation engine 12, and the flow control valve may be the intake flow control valve 18 and/or the exhalation flow control valve 19.

In the process of pressurizing to the preset target pressure value, different strategies may be selected. In one embodiment, the pressure may be directly increased to the target pressure value, which is simple and direct and is suitable for scenarios where the pressure setting is completed quickly. In other embodiments, staged pressurization is also possible. For example, the pressure is first raised to a lower first-level pressure value, and then the pressurization is paused to wait for the pressure to stabilize. After confirming that the first-level pressure value is stable, the pressure continues to be increased step by step to the target pressure value. Staged pressurization helps ensure the stability of the system at different pressure stages while providing accurate measurement conditions for a compliance characteristics of the reservoir bag. The above is not limited in embodiments of the present disclosure.

Step 202: Determining a compliance characteristic of the reservoir bag.

The internal pressure of the reservoir bag is monitored in the process of pressurizing the circuit, and pressure-versus-time data points are recorded as the pressure within the reservoir bag increases to form a curve of the internal pressure of the reservoir bag versus time. The curve of the internal pressure of the reservoir bag versus time is obtained to determine a compliance of the reservoir bag, thereby obtaining the compliance characteristic of the reservoir bag.

The compliance of the reservoir bag refers to the expandability of the reservoir bag when subjected to an external force, such as inflation. It can qualitatively and intuitively reflect a degree of “softness” and “hardness” of the reservoir bag. The compliance may also be described more specifically in a quantitative manner. For example, it may be quantitatively represented by measuring a change in the volume of the reservoir bag under unit pressure change. That is, the compliance is the ratio of the volume change value of the reservoir bag to the pressure change value. The compliance of the reservoir bag is negatively correlated with the hardness of the reservoir bag, that is, the greater the compliance of the reservoir bag, the greater the volume change of the reservoir bag under unit pressure change, which generally means that the hardness of the reservoir bag is lower. On the contrary, the less the compliance of the reservoir bag, the smaller the volume change of the reservoir bag under the unit pressure change, which generally means that the hardness of the reservoir bag is higher. Alternatively, the compliance of the reservoir bag may also be described by the pressure change characteristics of the reservoir bag. For example, when the reservoir bag (or the gas circuit including the reservoir bag) is inflated at a constant rate, the compliance is described by a pressure change rate in the reservoir bag over time. A smaller pressure change rate over time represents a greater volume change of the reservoir bag after a unit amount of gas is filled, that is, the lower the hardness of the reservoir bag is, the greater the compliance is. On the contrary, a greater pressure change rate over time represents that the reservoir bag is less likely to deform and has less compliance.

The compliance characteristic includes the compliance (for example, the magnitude of the numerical value) of the reservoir bag, or includes a comparison result between the reservoir bag and a standard reservoir bag. The comparison result between the reservoir bag and the standard reservoir bag is a comparison result of a compliance magnitude and/or a compliance similarity between the reservoir bag and the standard reservoir bag, and the compliance similarity is a similarity between the compliance of the reservoir bag and the compliance of the preset standard reservoir bag. It should be noted that, for the definition of the standard reservoir bag and the manner for determination of the compliance of the standard reservoir bag, reference may be made to the related description in the following embodiments, and details are not introduced here.

Step 203: Stopping pressurizing the circuit, and determining a wait time of the circuit on the basis of the compliance characteristic of the reservoir bag.

When the pressure in the circuit reaches the target pressure value, a driving gas inlet valve is closed to cut off driving gas flowing through the circuit, and pressurizing the circuit is stopped. It waits for the pressure to stabilize to ensure uniform pressure distribution in the circuit. The wait time of the circuit is determined on the basis of the compliance characteristic of the reservoir bag, so as to monitor the pressure change in step 204.

The pressurization stage of the circuit refers to a stage in which the internal pressure reaches a preset target pressure value by pressurizing the reservoir bag. A leak calculation stage refers to a stage in which, after the pressurization stage, pressurizing the circuit is stopped, and a pressure drop of the circuit for a specific time due to leaks is observed and calculated.

The wait time of the circuit is a wait time of the leak calculation stage, also referred to as a measurement time interval. The wait time of the circuit is a period of time from a first moment when the wait time starts, i.e. when measurement of the pressure change in the circuit starts, to a second moment when the wait time ends, i.e. when the measurement of the pressure change in the circuit ends.

In a leak test algorithm, the measurement of the pressure change in the circuit starts after a preset stabilization time period elapses from the time when the pressure in the circuit reaches the target pressure value, that is, the first moment may be a moment after the pressure in the circuit reaches the target pressure value, plus the preset stabilization time period. The stabilization time period refers to a duration required from the time when the pressure in the circuit reaches the target pressure value to the time when the circuit reaches a stable state. The stable state of the circuit is used to indicate a state where the pressure, volume or other related parameters inside of the circuit reach stability. For example, the preset stabilization time period is 3.5 seconds. The specific value of the stabilization time period is not limited in the embodiments of the present disclosure.

Optionally, two strategies may be selected to determine the wait time of the circuit on the basis of the compliance characteristic of the reservoir bag. One is to directly determine the wait time of the circuit according to the compliance of the reservoir bag, and the other is to adjust a standard wait time corresponding to the standard reservoir bag according to the comparison result (including the compliance magnitude and/or the compliance similarity) between the reservoir bag and the standard reservoir bag, thereby obtaining the wait time of the circuit.

It should be noted that the manner for determination of the wait time is not limited to the above method, and the wait time may be determined by flexibly selecting or combining different methods according to specific situations and requirements. For related details of the manner for determination of the wait time, reference may be made to related descriptions in the following embodiments, and they are not introduced here.

Step 204: Determining a change in gas pressure in the circuit before and after the wait time, thereby testing whether the circuit is leaking.

By analyzing the change in the gas pressure of the circuit before and after the wait time, whether there is leakage in the circuit is accurately detected. This testing process provides a scientific approach to determination of the seal integrity of the circuit on the basis of the measurement of the pressure change.

In this process, on the basis of the wait time determined in the previous step, which starts from the first moment and ends at the second moment, an initial gas pressure of the circuit is measured when the wait time starts (i.e., the first moment) and recorded as a first pressure value. At the end (i.e., the second moment) of the wait time, the gas pressure of the circuit is measured again and recorded as a second pressure value. By calculating the difference between the first pressure value and the second pressure value, an amount of gas pressure change of the circuit before and after the wait time can be obtained, that is, the amount of gas pressure change is the difference between the first pressure value and the second pressure value. This amount of gas pressure change reflects the change in the gas pressure in the circuit before and after the wait time. If the first pressure value is greater than the second pressure value, this indicates that the gas pressure in the circuit has dropped during the wait time, which may be due to a leak. In the above manner, a qualitative analysis result can be simply and quickly provided for the determination of the circuit leak.

Optionally, according to the measured compliance of the reservoir bag, the wait time, and the amount of gas pressure change of the circuit before and after the wait time, a leak rate of the circuit is determined. The leak rate is used to indicate whether the circuit is leaking and the severity of the leak. The leak rate is an indicator for the measure of the severity of a leak, and combines the compliance, pressure change, and time factors to provide a comprehensive evaluation. Illustratively, the ratio of the amount of gas pressure change of the circuit before and after the wait time to the wait time is determined, and the product of the ratio and the measured compliance of the reservoir bag is determined to be the leak rate of the circuit. In this way, an accurate quantitative analysis result can be provided for the degree of leakage of the circuit. It should be noted that the specific manner for calculation of the leak rate of the circuit is not limited in the embodiments of the present disclosure.

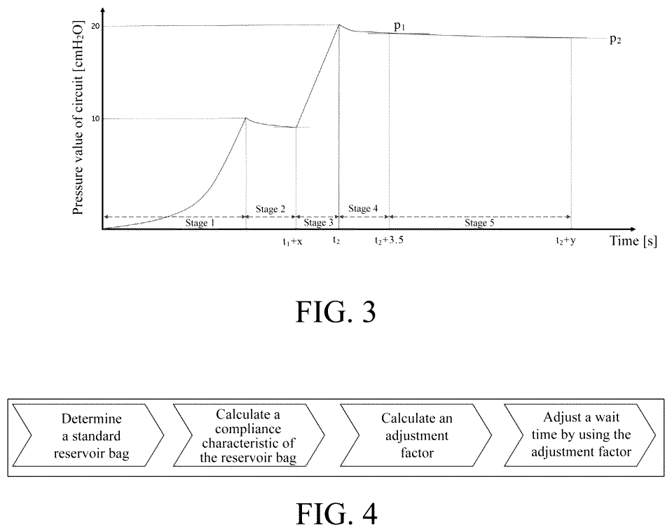

In an illustrative example, as shown in FIG. 3, FIG. 3 shows a curve graph of changes in a pressure value of a circuit versus time during testing provided by an exemplary embodiment of the present disclosure. It can be seen that the method for leak testing in a circuit of a medical device provided by the embodiments of the present disclosure includes the following stages: stage 1, pressurizing a circuit of a medical device to 10 centimeter water column (cmH₂O); stage 2, waiting for a period of time until the pressure is stable; stage 3, pressurizing the circuit to 20 centimeter water column (cmH₂O) and determining a compliance characteristic of the circuit; stage 4, stopping pressurizing the circuit, and waiting for 3.5 seconds for the pressure to stabilize; and stage 5, determining a wait time of the circuit on the basis of the compliance characteristic of the reservoir bag, and monitoring a change in gas pressure in the circuit before and after the wait time, thereby testing whether the circuit is leaking. For example, according to the compliance C of the reservoir bag, the wait time Δt, and the amount of gas pressure change Δp of the circuit before and after the wait time, the leak rate L of the circuit may be determined by the following formula:

$$L = C \frac{Δ p}{Δ t} = C \frac{{p}_{1} - {p}_{2}}{Δ t}$$

where p₁ is a first pressure value of the circuit when the wait time starts (i.e., t₂+3.5), and p₂ is a second pressure value of the circuit when the wait time ends (i.e. t₂+y), y being a numerical value greater than 3.5.

In summary, the embodiments of the present disclosure provide a method for leak testing in a circuit of a medical device. By pressurizing a circuit, a compliance characteristic of a reservoir bag included in the circuit is determined; after stopping pressurizing the circuit, on the basis of the compliance characteristic of the reservoir bag, a wait time of the circuit is determined; and a change in gas pressure in the circuit before and after the wait time is determined, thereby testing whether the circuit is leaking. That is, by adapting to the compliance characteristics of different reservoir bags, accurate testing for reservoir bags of various types and sizes is achieved, thereby greatly improving the accuracy and reliability of the test. The adaptive design of this method not only avoids the need for additional configuration of system software, but also ensures the robustness of the test results. For a user, the user can freely select the required reservoir bag, and the test can be performed without any additional configuration before using the specific reservoir bag, which ensures the safety and performance of the medical device.

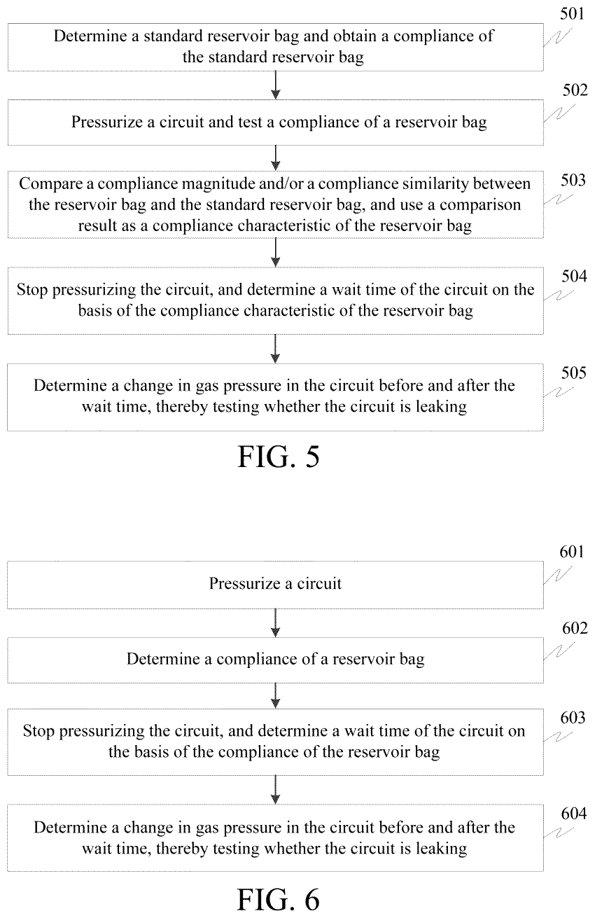

In a possible implementation, in the method for leak testing in a circuit of a medical device provided by the embodiments of the present disclosure, the method is performed by comparing the compliance magnitude and/or a compliance similarity between the reservoir bag being used and a preset standard reservoir bag. FIG. 4 provides an intuitive exemplary flowchart. In this solution, a standard reservoir bag is first determined as a reference, and then a compliance characteristic of a reservoir bag is determined by comparing a compliance magnitude and/or a compliance similarity between the reservoir bag and the standard reservoir bag. Next, an adjustment factor is calculated on the basis of the compliance characteristic of the reservoir bag, thereby adjusting a wait time of a circuit by using the adjustment factor. In order to further explain the detailed flows of this test method, and more clearly understand the specific content and purpose of each step of operation, reference may be made to FIG. 5. FIG. 5 shows a flowchart of a method for leak testing in a circuit of a medical device provided by another exemplary embodiment of the present disclosure. This embodiment is illustrated by taking the method being applied to the medical device as an example. The method includes the following steps:

Step 501: Determining a standard reservoir bag and obtaining the compliance of the standard reservoir bag.

To ensure consistency and repeatability of test results, a standard reservoir bag is pre-selected and its characteristics are defined in software. This step involves conventional leak testing in circuits of reservoir bags commonly used in hospitals and comparison among them to determine which reservoir bag provides results with the greatest stability and least variance in the test.

The standard reservoir bag has the property of providing consistent results under different test conditions, ensuring that the test results are not affected by external factors. For example, in a number of tests, the standard reservoir bag is one among a plurality of candidate reservoir bags that provides the smallest leak result, which means that the performance of the standard reservoir bag in repeated tests should be stable and predictable. The standard reservoir bag should represent the type of reservoir bags commonly used in hospitals, so that the test results can reflect the actual use. The standard reservoir bag is selected from a plurality of candidate reservoir bags through a series of scientific methods and tests, and provides the best test performances among all the candidate reservoir bags. The characteristics of the standard reservoir bag should be precisely defined in software for use in the process of automated testing, ensuring that each test is performed with the same criteria. In other words, the standard reservoir bag may be understood as a reservoir bag having a constant compliance. Thus, this can provide a consistent standard for testing at different times and reservoir bags with different compliances under test. An exemplary description of the selection of the standard reservoir bag will be given below.

The step of selecting the standard reservoir bag may include: selecting a plurality of candidate reservoir bags that are most commonly used in hospitals. Each candidate reservoir bag is tested multiple times (e.g., 10 times), and the test results are compared to select the standard reservoir bag. The selected standard reservoir bag is a reservoir bag that shows the least leak variance in the multiple tests.

The step of defining the characteristics in the software may include: testing the selected standard reservoir bag multiple times (e.g., 10 times). In each test, a pressure change rate of the standard reservoir bag is calculated at a fixed time interval (for example, every 500 milliseconds), and the pressure change rate of the standard reservoir bag refers to the change in the internal pressure of the standard reservoir bag in the process of pressurizing. Specifically, the pressure change rate of the standard reservoir bag is determined by measuring the increase in pressure of the standard reservoir bag during the time period from the start of pressurization to the end of pressurization. The pressure change rate of the standard reservoir bag is the pressure change rate of the standard reservoir bag over time during the process of pressurizing, that is, the ratio of the pressure change value to the time change value, or the pressure change rate of the standard reservoir bag is the pressure change rate of the standard reservoir bag along with the volume during the process of pressurizing, that is, the ratio of the pressure change value to the volume change value of the reservoir bag (i.e., an inverse of the compliance). Then, multiple pressure change rates of the standard reservoir bag obtained in the multiple tests are averaged to obtain a plurality of standard data sets of the standard reservoir bag. Taking the pressure change rate being the pressure change rate over time as an example, each standard data set includes a standard pressure value and a corresponding average value (i.e., standard value) of pressure change rates over time. The compliance can be obtained through the pressure change rate, and the compliances of the standard reservoir bag obtained in multiple tests are averaged to obtain a final compliance of the standard reservoir bag, so as to reduce the influence of random errors, and improve the stability and repeatability of data. In the software, the pressure change rate and compliance of the standard reservoir bag obtained by averaging after multiple tests are defined as constants, and these average values are used as a reference in the software to evaluate the performances of other reservoir bags to be tested.

Step 502: Pressurizing a circuit and testing a compliance of a reservoir bag.

Pressurizing the circuit is the basis for creating a test environment. By this step, it is ensured that the pressure in the circuit reaches a level suitable for testing. Next, the compliance of the reservoir bag is tested, which is a key indicator to evaluate the ability of the reservoir bag to respond to pressure changes. During the process of pressurizing, the change in the internal pressure of the reservoir bag over time is monitored, a curve of change in the pressure is obtained to test the compliance of the reservoir bag, and the compliance of the reservoir bag is stored in a variable, wherein the compliance is the ratio of the volume change value of the reservoir bag to the pressure change value. The volume change value of the reservoir bag may be determined by a flow sensor, that is, the flow sensor measures the flow of gas entering and leaving the reservoir bag, thereby estimating the volume change value of the reservoir bag.

Step 503: Comparing a compliance magnitude and/or a compliance similarity between the reservoir bag and the standard reservoir bag, and using a comparison result as a compliance characteristic of the reservoir bag.

Optionally, the compliance magnitude between the reservoir bag and the standard reservoir bag is determined according to the compliance of the reservoir bag and the compliance of the standard reservoir bag; and/or, the compliance similarity between the reservoir bag and the standard reservoir bag is determined according to the pressure change rate of the reservoir bag over time and the pressure change rate of the standard reservoir bag over time. After the compliance similarity is determined, the determined compliance similarity may be stored in a variable, and the variable may be used for subsequent analysis and evaluation.

The compliance similarity between the reservoir bag and the standard reservoir bag is inferred by comparing the pressure change rates over time of the reservoir bag and the standard reservoir bag. The principle is as follow: For a relatively hard reservoir bag, the compliance thereof is less, and correspondingly, the pressure rise caused by the same pressurization time is larger, that is, the pressure change rate over time is larger. On the contrary, for a relatively soft reservoir bag, the compliance thereof is greater, and correspondingly, the pressure rise caused by the same pressurization time is smaller, that is, the pressure change rate over time is smaller. Therefore, the pressure change rate over time can also reflect the degree of softness or hardness of the reservoir bag, and has a certain correlation with the compliance, and the similarity between the compliances can be reflected by the similarity between the pressure change rates over time. Moreover, the comparison between the reservoir bag and the standard reservoir bag is performed by using different data such as the compliance and the pressure change rate over time, which can avoid the result deviation caused by the error of the same type of data and improve the accuracy of the comparison result.

Optionally, the compliance magnitude between the reservoir bag and the standard reservoir bag is the difference between the compliance of the reservoir bag and the compliance of the standard reservoir bag. The compliance is negatively correlated with the hardness of the reservoir bag. When the compliance of the reservoir bag is less than the compliance of the standard reservoir bag, the compliance magnitude is determined to be a negative value, indicating that the hardness of the reservoir bag is higher than the hardness of the standard reservoir bag. When the compliance of the reservoir bag is greater than the compliance of the standard reservoir bag, the compliance magnitude is determined to be a positive value, indicating that the hardness of the reservoir bag is lower than the hardness of the standard reservoir bag. When the compliance of the reservoir bag is equal to the compliance of the standard reservoir bag, the compliance magnitude is determined to be zero, indicating that the hardness of the reservoir bag is equal to the hardness of the standard reservoir bag.

Optionally, during the pressurization stage of the reservoir bag, the pressure change rate of the reservoir bag over time is measured and recorded at a fixed time interval (for example, every 500 milliseconds), and the measured value is stored in a vector in the memory for comparison with the data of the standard reservoir bag. That is, the pressure change rate of the reservoir bag over time includes a plurality of measured values, the pressure change rate of the standard reservoir bag over time includes a plurality of standard values, and determining the compliance similarity between the reservoir bag and the standard reservoir bag according to the pressure change rate of the reservoir bag over time and the pressure change rate of the standard reservoir bag over time includes: determining standard values respectively corresponding to the plurality of measured values from among the plurality of standard values; and determining the compliance similarity between the reservoir bag and the standard reservoir bag by using a preset similarity calculation model according to the plurality of measured values and the standard values respectively corresponding to the plurality of measured values.

Optionally, a plurality of standard data sets of the standard reservoir bag are pre-stored, each standard data set includes a standard pressure value and a corresponding standard value of the pressure change rate over time, and determining the standard values respectively corresponding to the plurality of measured values from among the plurality of standard values may include: for each measured value, determining a first pressure value corresponding to the measured value, finding a standard pressure value numerically closest to the first pressure value in the plurality of pre-stored standard data sets, and determining a standard value of the pressure change rate over time corresponding to the standard pressure value.

The similarity calculation model is a mathematical or empirical model, and the similarity calculation model is used for indicating a mapping relationship among the plurality of measured values, the plurality of standard values, and the compliance similarity. The similarity calculation model is a mathematical or empirical model for converting the plurality of measured values and the plurality of standard values into the compliance similarity. In the case of reservoir bag tests, the similarity calculation model defines a quantitative relationship among the plurality of measured values, the plurality of standard values, and the compliance similarity. This similarity calculation model may be built on the basis of historical data, experimental results, or theoretical analysis, with the purpose of providing an accurate and repeatable method to determine the compliance similarity between the reservoir bag and the standard reservoir bag.

In a possible implementation, the compliance similarity between the reservoir bag and the standard reservoir bag is determined by using a generalized Jaccard similarity coefficient formula. The formula is as follows:

$${J}_{g} ( a , b ) = \frac{{\sum }_{i} min ( {a}_{i} , {b}_{i} )}{{\sum }_{i} max ( {a}_{i} , {b}_{i} )}$$

where a and b are two vectors, a=[a₁, a₂, . . . aₙ], b=[b₁, b₂, . . . bₙ], aᵢ is a measured value of a pressure change characteristic (e.g., a pressure change rate) of the reservoir bag at a time point i, bᵢ is a standard value of the standard reservoir bag corresponding to aᵢ, i is a positive integer, the value of i is in the range of 1 to n, n is a positive integer greater than 1, and J^{g}(a, b) is the compliance similarity between the reservoir bag and the standard reservoir bag.

It should be noted that, in addition to the above generalized Jaccard similarity coefficient formula, other similarity calculation methods, such as Euclidean distance and cosine similarity, may also be considered to adapt to different evaluation requirements and scenarios. The above is not limited in embodiments of the present disclosure.

When the compliance similarity between the reservoir bag and the standard reservoir bag is calculated, additional sampled values may be added to improve the accuracy of evaluation. These sampled values may include a pressure change rate from a moment when the pressure in the circuit reaches the target pressure value to the first moment, i.e. the moment when the measurement of the pressure change in the circuit starts. This method provides a more comprehensive data set that helps to more accurately evaluate the performance of the reservoir bag.

Step 504: Stopping pressurizing the circuit, and determining a wait time of the circuit on the basis of the compliance characteristic of the reservoir bag.

When the pressure in the circuit reaches the target pressure value, pressurizing the circuit is stopped. In an example, the standard wait time corresponding to the standard reservoir bag may be adjusted according to the compliance characteristic of the reservoir bag, that is, the comparison result of the compliance magnitude and/or the compliance similarity between the reservoir bag and the standard reservoir bag, and the adjusted standard wait time may be determined to be the wait time of the circuit. The manner for determination of the wait time of the circuit may be determination based only on the compliance similarity between the reservoir bag and the standard reservoir bag, may be determination based only on the compliance magnitude between the reservoir bag and the standard reservoir bag, or may be determination by comprehensively considering the compliance magnitude and the compliance similarity between the reservoir bag and the standard reservoir bag. The three manners for determination will be further introduced below separately.

In a possible implementation, determining the wait time of the circuit only on the basis of the compliance similarity between the reservoir bag and the standard reservoir bag includes the following steps: according to the compliance similarity between the reservoir bag and the standard reservoir bag, determining a corresponding adjustment factor by using a preset mapping model, the mapping model being used for indicating a correspondence between the compliance similarity and the adjustment factor; and adjusting the standard wait time corresponding to the standard reservoir bag according to the adjustment factor, and determining the adjusted standard wait time to be the wait time of the circuit.

The standard wait time is a preset reference time value of the standard reservoir bag, and is used as a reference value of the wait time of the leak test in a circuit. The standard wait time is typically based on extensive testing and validation to ensure the accuracy and consistency of testing under general conditions.

The mapping model is a mathematical or empirical model for mapping or converting the compliance similarity between the reservoir bags into the adjustment factor of the wait time. In the case of reservoir bag tests, the mapping model defines a mapping relationship between the compliance similarity and the adjustment factor. This model may be built on the basis of historical data, experimental results, or theoretical analysis, with the purpose of providing an accurate and repeatable method to predict and adjust the standard wait time. Optionally, the compliance similarity between the reservoir bag and the standard reservoir bag is positively correlated with the adjustment factor, that is, the greater the compliance similarity between the reservoir bag and the standard reservoir bag is, the greater the numerical value of the adjustment factor is.

In this implementation, the adjustment factor is an output of the mapping model, and is a numerical value used to adjust the standard wait time. Optionally, the product of the adjustment factor and the standard wait time is determined to be the wait time of the circuit. For example, if the standard wait time is 10 seconds and the adjustment factor is 0.8, the wait time of the circuit is 10*0.8=8 seconds.

In another possible implementation, determining the wait time of the circuit only on the basis of the compliance magnitude between the reservoir bag and the standard reservoir bag includes the following steps: when the compliance of the reservoir bag is less than the compliance of the standard reservoir bag, that is, when the compliance magnitude indicates that the hardness of the reservoir bag is higher than the hardness of the standard reservoir bag, shortening the standard wait time according to the adjustment factor; when the compliance of the reservoir bag is greater than the compliance of the standard reservoir bag, that is, when the compliance magnitude indicates that the hardness of the reservoir bag is lower than the hardness of the standard reservoir bag, extending the standard wait time according to the adjustment factor; and when the compliance of the reservoir bag is equal to the compliance of the standard reservoir bag, that is, when the compliance indicates that the hardness of the reservoir bag is equal to the hardness of the standard reservoir bag, keeping the standard wait time unchanged. After the standard wait time is adjusted, the adjusted standard wait time is determined to be the wait time of the circuit.

In this implementation, the adjustment factor may be a preset value, and the adjustment factor may be set by default or customized. The adjustment factor may be a preset numerical value or one of a plurality of preset numerical values, and is used to adjust the standard wait time. Optionally, when the compliance of the reservoir bag is less than the compliance of the standard reservoir bag, the difference between the standard wait time and a first adjustment value is determined to be the wait time of the circuit; and when the compliance of the reservoir bag is greater than the compliance of the standard reservoir bag, the sum of the standard wait time and the first adjustment value is determined to be the wait time of the circuit, wherein the first adjustment value is the product of the adjustment factor and the standard wait time. For example, the standard wait time is 10 seconds, the preset adjustment factor is 0.1, and when the compliance of the reservoir bag is less than the compliance of the standard reservoir bag, the wait time of the circuit is 10-10*0.1=9 seconds. When the compliance of the reservoir bag is greater than the compliance of the standard reservoir bag, the wait time of the circuit is 10+10*0.1=11 seconds.

In another possible implementation, determining the wait time of the circuit by comprehensively considering the compliance magnitude and the compliance similarity between the reservoir bag and the standard reservoir bag includes the following steps: when the compliance of the reservoir bag is less than the compliance of the standard reservoir bag, that is, when the compliance magnitude indicates that the hardness of the reservoir bag is higher than the hardness of the standard reservoir bag, according to the compliance similarity, determining a corresponding first adjustment factor by using a preset first mapping model, shortening the standard wait time according to the first adjustment factor, and determining the shortened standard wait time to be the wait time of the circuit; when the compliance of the reservoir bag is greater than the compliance of the standard reservoir bag, that is, when the compliance magnitude indicates that the hardness of the reservoir bag is lower than the hardness of the standard reservoir bag, according to the compliance similarity, determining a corresponding second adjustment factor by using a preset second mapping model, extending the standard wait time according to the second adjustment factor, and determining the extended standard wait time to be the wait time of the circuit, wherein the second mapping model is different from the first mapping model; and when the compliance of the reservoir bag is equal to the compliance of the standard reservoir bag, that is, when the compliance magnitude indicates that the hardness of the reservoir bag is equal to the hardness of the standard reservoir bag, keeping the standard wait time unchanged, and determining the standard wait time to be the wait time of the circuit.

The first mapping model and the second mapping model are each a mathematical or empirical model. The first mapping model is used to indicate a mapping relationship between the compliance similarity of the reservoir bag and the first adjustment factor, and the first mapping model is used to map or convert the compliance similarity of the reservoir bag into the first adjustment factor of the wait time. The second mapping model is used to indicate a mapping relationship between the compliance similarity of the reservoir bag and the second adjustment factor, the second mapping model is used to map or convert the compliance similarity of the reservoir bag into the second adjustment factor of the wait time, and the second mapping model is different from the first mapping model.

Optionally, the compliance similarity may be a positive number between 0 and 1, the first mapping model is used to indicate that the first adjustment factor is equal to the compliance similarity of the reservoir bag, and the first adjustment factor is a positive number between 0 and 1. The second mapping model includes a first sub-model or a second sub-model, the first sub-model is used to indicate that the second adjustment factor is the difference between 2 and the compliance similarity of the reservoir bag, the second sub-model is used to indicate that the second adjustment factor is the ratio between 1 and the compliance similarity of the reservoir bag, and the second adjustment factor is a positive number greater than 1. That is, the numerical value of the second adjustment factor obtained by the second sub-model may be much greater than the numerical value of the second adjustment factor obtained by the first sub-model, so as to produce a greater adjustment effect. The above specific numerical values are only examples, and may be set as needed.

The second mapping model may be selected in the following manner: When the compliance of the reservoir bag is greater than the compliance of the standard reservoir bag and the compliance similarity is greater than or equal to a preset similarity threshold (that is, when there is a smaller difference in the degree of softness or hardness between the reservoir bag and the standard reservoir bag), the second mapping model used is the first sub-model. When the compliance of the reservoir bag is greater than the compliance of the standard reservoir bag and the compliance similarity is less than the preset similarity threshold (that is, when there is a larger difference in the degree of softness or hardness between the reservoir bag and the standard reservoir bag), a model for more aggressive adjustment may be used, that is, the second mapping model used is the second sub-model. The manner for setting the first mapping model and the second mapping model is not limited in the embodiments of the present disclosure.

In this implementation, the adjustment factor (the first adjustment factor or the second adjustment factor) is a numerical value, and is used to adjust the standard wait time. In some embodiments, the product of the adjustment factor (the first adjustment factor or the second adjustment factor) and the standard wait time may be determined to be the wait time of the circuit. For example, if the standard wait time is 10 seconds and the second adjustment factor is 1.5, the wait time of the circuit is 10*1.5=15 seconds.

In some other embodiments, the difference between the second adjustment value and the preset stabilization time period may be determined to be the wait time of the circuit, and the second adjustment value is the product of the adjustment factor (that is, the first adjustment factor or the second adjustment factor) and the standard total wait time (that is, the sum of the preset stabilization time period and the standard wait time). For example, the moment when the pressure in the circuit reaches the target pressure value is t₂, the preset stabilization time period is 3.5 seconds, the moment when the measurement of the pressure change in the circuit starts (i.e., the first moment) is t₂+3.5, the second moment when the measurement of the pressure change in the circuit ends (i.e., the second moment) is t₂+13.5*adjustment factor, and the wait time of the circuit is the difference between the second moment and the first moment, i.e., 13.5*adjustment factor−3.5.

Step 505: Determining a change in gas pressure in the circuit before and after the wait time, thereby testing whether the circuit is leaking.

According to the measured compliance of the reservoir bag, the wait time, and the amount of gas pressure change of the circuit before and after the wait time, a leak rate of the circuit is determined. The leak rate is used to indicate whether the circuit is leaking and the severity of the leak. For related details, reference may be made to the related descriptions in the foregoing embodiments, and the details are not described here again.

In summary, in the method for leak testing in a circuit of a medical device provided by the embodiments of the present disclosure, in one aspect, the compliance characteristics of the reservoir bag may be obtained by testing the compliance of the reservoir bag and comparing the compliance of the reservoir bag with the compliance of the standard reservoir bag. The technical effect is that a more precise wait time can be set for the characteristics of a specific reservoir bag, thereby improving the accuracy and efficiency of leak detection. In another aspect, by using the preset mapping model, the adjustment factor is determined according to the compliance similarity between the reservoir bag and the standard reservoir bag, and then the standard wait time is adjusted. The technical effect is that the adaptive adjustment for reservoir bags with different compliances can be achieved, optimizing the test process. In another aspect, the wait time is intelligently adjusted according to the compliance magnitude of the reservoir bag. If the hardness of the reservoir bag is higher than that of the standard reservoir bag, the wait time is shortened; and if the hardness is lower than that of the standard reservoir bag, the wait time is extended. The technical effect is that the test method is more flexible and can adapt to reservoir bags with different hardness. In another aspect, when the hardness of the reservoir bag is higher or lower than that of the standard reservoir bag, different mapping models are used to determine the adjustment factor, thereby adjusting the wait time. The technical effect is that the adaptability to reservoir bags with different hardness and the accuracy of testing are improved. In another aspect, the internal pressure of the reservoir bag is monitored during the process of pressurizing, and the curve of the pressure versus time is obtained to test the compliance of the reservoir bag. The technical effect is that the status of the reservoir bag can be monitored in real time, providing data support for compliance testing. In another aspect, by comparing the pressure change rates of the reservoir bag and the standard reservoir bag, the compliance similarity is determined by using the similarity calculation model. The technical effect is that the similarity between the reservoir bag and the standard reservoir bag can be quantified, providing a basis for subsequent testing. In another aspect, the compliance is negatively correlated with the hardness of the reservoir bag, and the greater the compliance is, the lower the hardness is, and vice versa. The technical effect is that the hardness characteristic of the reservoir bag can be directly determined by the magnitude of the compliance. In another aspect, the method for testing is applicable to a ventilator device or an anesthesia machine device, which increases the application range and practicability of the method for testing.

In another possible implementation, in the method for leak testing in a circuit of a medical device provided by the embodiments of the present disclosure, the wait time is determined directly on the basis of the determination of the compliance of the reservoir bag, rather than the comparison with the standard reservoir bag. Referring to FIG. 6, FIG. 6 shows a flowchart of a method for leak testing in a circuit of a medical device provided by another exemplary embodiment of the present disclosure. This embodiment is illustrated by taking the method being applied to the medical device as an example. The method includes the following steps: step 601, pressurizing a circuit; step 602, determining a compliance of a reservoir bag; step 603, stopping pressurizing the circuit, and determining a wait time of the circuit on the basis of the compliance of the reservoir bag; step 604, determining a change in gas pressure in the circuit before and after the wait time, thereby testing whether the circuit is leaking.

The manner for determining the wait time of the circuit on the basis of the compliance of the reservoir bag in step 603 includes, but is not limited to, the following possible implementations.

In one possible implementation, the wait time of the circuit is predicted by using an experience-based formula according to the compliance of the reservoir bag. The formula used therein may be based on past experimental data and practical operation experiences, and is used to indicate a mapping relationship between the compliance of the reservoir bag and the wait time of the circuit.

In another possible implementation, a pre-trained target prediction model is invoked to output the wait time of the circuit according to the compliance of the reservoir bag, and the target prediction model is used to indicate the mapping relationship between the compliance of the reservoir bag and the wait time of the circuit. A training process of the target prediction model may include: training a preset original parameter model on historical data to obtain the target prediction model, wherein the historical data includes a plurality of sample sets, each sample set includes compliances of sample reservoir bags and the actual wait time of the circuit, and the original parameter model may be a model using a neural network or other machine learning algorithms.

It should be noted that, for related details of other steps in this embodiment, reference may be made to the related descriptions in the foregoing embodiments, and the details are not described here again.

In summary, in the method for leak testing in a circuit of a medical device provided by the embodiments of the present disclosure, the wait time of the circuit is also determined by directly analyzing the compliance of the reservoir bag, thereby determining whether there is a leak in the circuit. The step of comparing with the standard reservoir bag is omitted, the testing process is simplified, the total time required for testing is reduced, and the testing efficiency is improved. The generality and flexibility of the method for testing are further improved without relying on the comparison with the standard reservoir bag.

The embodiments of the present disclosure further provide an electronic device, including: a processor; a memory for storing processor-executable instructions, wherein the processor is configured to, when executing the instructions stored in the memory, implement the foregoing method.

The embodiments of the present disclosure further propose a computer-readable storage medium, having computer program instructions stored thereon, wherein the computer program instructions, when executed by a processor, implement the foregoing method. The computer-readable storage medium may be a volatile computer-readable storage medium or a non-volatile computer-readable storage medium.

The embodiments of the present disclosure further propose a computer program product, including computer-readable codes or a non-volatile computer-readable storage medium loaded with computer-readable codes, wherein when the computer-readable codes are run in a processor of an electronic device, the processor in the electronic device performs the foregoing method.

The present disclosure may be a system, a method and/or a computer program product. The computer program product may include a computer-readable storage medium, having computer-readable program instructions thereon for causing a processor to implement various aspects of the present disclosure.

The computer-readable storage medium can be a tangible device that can hold and store instructions used by an instruction execution device. The computer-readable storage medium may be, for example, but is not limited to an electrical storage device, a magnetic storage device, an optical storage device, an electromagnetic storage device, a semiconductor memory device, or any suitable combination thereof. More specific examples (a non-exhaustive list) of the computer-readable storage medium include: a portable computer disk, a hard disk, a random access memory (RAM), a read only memory (ROM), a erasable programmable read only memory (EPROM or flash memory), a static random access memory (SRAM), a portable compact disc read-only memory (CD ROM), a digital versatile disk (DVD), a memory stick, a floppy disk, mechanical coding equipment, such as a punch card with instructions stored thereon or a structure of bumps within recessions, and any suitable combination thereof. The computer-readable storage medium used herein is not interpreted as transient signals themselves, such as radio waves or other freely propagated electromagnetic waves, electromagnetic waves propagated through a waveguide or other transmission media (e.g., light pulses passing through a fiber optic cable), or electrical signals transmitted through electric wires.

The computer-readable program instructions described herein may be downloaded from a computer-readable storage medium to various computing/processing devices or downloaded to an external computer or external storage device via a network such as the Internet, a local area network, a wide area network and/or a wireless network. The network may include copper transmission cables, fiber transmission, wireless transmission, routers, firewalls, switches, gateway computers, and/or edge servers. A network adapter card or a network interface in each computing/processing device receives computer-readable program instructions from the network and forwards the computer-readable program instructions, for storing them in a computer-readable storage medium in each computing/processing device.

Computer program instructions for performing the operations of the present disclosure can be assembly instructions, instruction set architecture (ISA) instructions, machine instructions, machine related instructions, microcode, firmware instructions, state setting data, or source code or object code written in any combination of one or more programming languages, the programming language including object oriented programming languages such as Smalltalk, C++ and the like, and conventional procedural programming languages such as the “C” language or similar programming languages. The computer-readable program instructions can be executed entirely or partly on a user computer, executed as a stand-alone software package, executed partly on a user computer and partly on a remote computer, or executed entirely on a remote computer or server. In the case of a remote computer, the remote computer may be connected to the user's computer through any kind of network, including a local area network (LAN) or a wide area network (WAN). Alternatively, it can be connected to an external computer (for example, using an Internet service provider to connect via the Internet). In some embodiments, an electronic circuit, for example, a programmable logic circuit, a field-programmable gate array (FPGA), or a programmable logic array (PLA), may execute the computer-readable program instructions by utilizing state information of the computer-readable program instructions to personalize the electronic circuit, in order to implement various aspects of the present disclosure

The aspects of the present disclosure are described herein with reference to the flowcharts and/or block diagrams of the methods, apparatuses (systems), and computer program products according to the embodiments of the present disclosure. It should be understood that each block of the flowcharts and/or block diagrams and combinations of various blocks in the flowcharts and/or block diagrams can be implemented by computer-readable program instructions.

These computer-readable program instructions may be provided to a processor of a general-purpose computer, a special-purpose computer, or other programmable data processing apparatuses, to produce a machine, so that these instructions, when executed by the processor of the computer or other programmable data processing apparatuses, produce an apparatus for implementing the functions/actions specified in one or more blocks of the flowcharts and/or block diagrams. Also, these computer-readable program instructions may be stored in a computer-readable storage medium. These instructions cause a computer, a programmable data processing device, and/or other devices to work in a specific manner. Thus, the computer-readable medium storing the instructions includes an artifact, including instructions that implement various aspects of the functions/actions specified in one or more the flowcharts and/or block diagrams.

The computer-readable program instructions may also be loaded onto a computer, other programmable data processing apparatuses, or other devices, such that the computer, other programmable data processing apparatuses or other devices perform a series of operational steps, to generate a computer-implemented process, such that the functions/actions specified in one or more of the flowcharts and/or block diagrams are implemented by the instructions executed on the computer, other programmable data processing apparatuses, or other devices.

The flowcharts and block diagrams in the accompanying drawings illustrate system architectures, functions, and operations of possible implementations of the system, method, and computer program product according to a plurality of embodiments of the present disclosure. In this regard, each block in the flowcharts or block diagrams may represent a portion of a module, program segment, or instruction that contains one or more executable instructions for implementing the specified logical functions. In some alternative implementations, the functions denoted in the blocks can also occur in a different order than that illustrated in the drawings. For example, two consecutive blocks can actually be performed substantially in parallel, and sometimes can also be performed in a reverse order, depending upon the functions involved. It should also be noted that each block of the block diagrams and/or flowcharts, and combinations of blocks in the block diagrams and/or flowcharts can be implemented in a dedicated hardware-based system that performs the specified function or action, or can be implemented by a combination of dedicated hardware and computer instructions.

The embodiments of the present disclosure have been described above. The foregoing description is illustrative rather than limiting, and is not limited to the disclosed embodiments. Many modifications and variations are apparent to those of ordinary skill in the art without departing from the scope and spirit of the embodiments illustrated. The selection of terms used herein is intended to best explain the principles and practical applications of the embodiments, or improvements to the techniques on the market, or to enable others of ordinary skill in the art to understand the embodiments disclosed herein.

1. A method for leak testing in a circuit of a medical device, the circuit comprising a reservoir bag, characterized in that the method comprises:
pressurizing the circuit;
determining a compliance characteristic of the reservoir bag;
stopping pressurizing the circuit, and determining a wait time of the circuit on the basis of the compliance characteristic of the reservoir bag; and
determining a change in gas pressure in the circuit before and after the wait time, thereby testing whether the circuit is leaking.

2. The method according to claim 1, wherein determining the compliance characteristic of the reservoir bag comprises:
testing a compliance of the reservoir bag;
obtaining a compliance of a standard reservoir bag;
comparing a compliance magnitude and/or a compliance similarity between the reservoir bag and the standard reservoir bag, and using a comparison result as the compliance characteristic of the reservoir bag.

3. The method according to claim 2, wherein determining the wait time of the circuit on the basis of the compliance characteristic of the reservoir bag comprises:
obtaining a standard wait time corresponding to the standard reservoir bag;
according to the compliance similarity between the reservoir bag and the standard reservoir bag, determining a corresponding adjustment factor by using a preset mapping model, the mapping model being used for indicating a correspondence between the compliance similarity and the adjustment factor; and
adjusting the standard wait time according to the adjustment factor, and determining the adjusted standard wait time to be the wait time of the circuit.

4. The method according to claim 2, wherein determining the wait time of the circuit on the basis of the compliance characteristic of the reservoir bag comprises:
obtaining a standard wait time corresponding to the standard reservoir bag, adjusting the standard wait time according to an adjustment factor, and determining the adjusted standard wait time to be the wait time of the circuit, and:
when the compliance magnitude indicates that a hardness of the reservoir bag is higher than a hardness of the standard reservoir bag, shortening the standard wait time according to the adjustment factor;
when the compliance magnitude indicates that a hardness of the reservoir bag is lower than a hardness of the standard reservoir bag, extending the standard wait time according to the adjustment factor; and
when the compliance magnitude indicates that the hardness of the reservoir bag is equal to the hardness of the standard reservoir bag, keeping the standard wait time unchanged.

5. The method according to claim 2, wherein determining the wait time of the circuit on the basis of the compliance characteristic of the reservoir bag comprises:
obtaining a standard wait time corresponding to the standard reservoir bag;
when the compliance magnitude indicates that a hardness of the reservoir bag is higher than a hardness of the standard reservoir bag, according to the compliance similarity, determining a corresponding first adjustment factor by using a preset first mapping model, shortening the standard wait time according to the first adjustment factor, and determining the shortened standard wait time to be the wait time of the circuit;
when the compliance magnitude indicates that a hardness of the reservoir bag is lower than a hardness of the standard reservoir bag, according to the compliance similarity, determining a corresponding second adjustment factor by using a preset second mapping model, extending the standard wait time according to the second adjustment factor, and determining the extended standard wait time to be the wait time of the circuit, wherein the second mapping model is different from the first mapping model; and
when the compliance magnitude indicates that the hardness of the reservoir bag is equal to the hardness of the standard reservoir bag, keeping the standard wait time unchanged, and determining the standard wait time to be the wait time of the circuit.

6. The method according to claim 2, wherein testing the compliance of the reservoir bag comprises:
monitoring an internal pressure of the reservoir bag in the process of pressurizing the circuit; and
obtaining a curve of change in the internal pressure of the reservoir bag versus time to test the compliance of the reservoir bag.

7. The method according to claim 6, wherein comparing the compliance magnitude and/or the compliance similarity between the reservoir bag and the standard reservoir bag comprises:
determining the compliance magnitude between the reservoir bag and the standard reservoir bag according to the compliance of the reservoir bag and the compliance of the standard reservoir bag; and/or,
determining the compliance similarity between the reservoir bag and the standard reservoir bag according to a pressure change rate of the reservoir bag over time and a pressure change rate of the standard reservoir bag over time.

8. The method according to claim 7, wherein the pressure change rate of the reservoir bag over time comprises a plurality of measured values, the pressure change rate of the standard reservoir bag over time comprises a plurality of standard values, and determining the compliance similarity between the reservoir bag and the standard reservoir bag according to the pressure change rate of the reservoir bag over time and the pressure change rate of the standard reservoir bag over time comprises:
determining standard values respectively corresponding to the plurality of measured values from among the plurality of standard values; and
determining the compliance similarity between the reservoir bag and the standard reservoir bag by using a preset similarity calculation model according to the plurality of measured values and the standard values respectively corresponding to the plurality of measured values.

9. The method according to claim 7, wherein the compliance is negatively correlated with a hardness of the reservoir bag, and:
when the compliance of the reservoir bag is less than the compliance of the standard reservoir bag, it is determined that the compliance magnitude indicates that a hardness of the reservoir bag is higher than a hardness of the standard reservoir bag;
when the compliance of the reservoir bag is greater than the compliance of the standard reservoir bag, it is determined that the compliance magnitude indicates that a hardness of the reservoir bag is lower than a hardness of the standard reservoir bag; and
when the compliance of the reservoir bag is equal to the compliance of the standard reservoir bag, it is determined that the compliance magnitude indicates the hardness of the reservoir bag is equal to the hardness of the standard reservoir bag.

10. The method according to any one of claim 1, wherein the medical device comprises a ventilator device or an anesthesia machine device.

11. A medical device, characterized by comprising:
a processor;
a memory for storing processor-executable instructions,
wherein the processor, when executing the instructions stored in the memory, is configured to:
pressurize the circuit;
determine a compliance characteristic of the reservoir bag;
stop pressurizing the circuit, and determining a wait time of the circuit on the basis of the compliance characteristic of the reservoir bag; and
determine a change in gas pressure in the circuit before and after the wait time, thereby testing whether the circuit is leaking.
